(19) United States
(12) Patent Application Publication
(10) Pub. No.: US 2017/0273820 A1
(43) Pub. Date: Sep. 28, 2017

(54) IUD INSERTION DEVICES, AND RELATED METHODS AND KITS THEREFOR

(71) Applicant: MEDICINES360, San Francisco, CA (US)

(72) Inventors: Rob DECKMAN, San Bruno, CA (US); Craig CARSON, Fremont, CA (US); Curt GUYER, Dublin, CA (US); David NETT, Malone, WI (US); Mitchell CREININ, Davis, CA (US)

(21) Appl. No.: 15/460,849

(22) Filed: Mar. 16, 2017

Related U.S. Application Data
(60) Provisional application No. 62/312,728, filed on Mar. 24, 2016.

(51) Int. Cl. A61F 6/18; A61M 31/00; A61F 6/14

(52) U.S. Cl. CPC A61F 6/18; A61F 6/144; A61M 31/00

(57) ABSTRACT

Disclosed are two-handed IUD insertion devices for positioning an intrauterine device (IUD) in the uterus which includes a plunger, a sheath, a grip and a handle where the axial movement of the plunger is controlled via features along the length of the plunger in communication with a grip feature.

### CROSS-REFERENCE

This application claims the benefit of U.S. Provisional Application No. 62/312,828, filed Mar. 24, 2016, entitled IUD Insertion Devices, and Related Methods and Kits Therefor, which application is incorporated herein by reference.

### BACKGROUND

Field of the Invention

The disclosure relates to intrauterine systems (IUS), intrauterine devices (IUDs), insertion devices, methods of use, and kits therefor.

Background of the Invention

An intrauterine device (IUD) is an object that, when placed in the uterus of a female, acts as a birth control device to prevent pregnancy. Two types of IUDs are commonly available, copper-containing devices and hormone-containing devices that release a progestogen. Hormone containing devices are considered to be a different form of birth control and may be distinguished in the literature by the term intrauterine system (IUS).

Copper IUDs work by negatively affecting the mobility of sperm and preventing the sperm from joining an egg. Additionally, the copper body positioned within the uterus irritates the lining of the uterus and uterine wall making it difficult for an embryo to plant in the wall if the egg is fertilized by the sperm. IUS devices, such as the hormonal IUD Mirena® (marketed by Bayer) reduce or prevent menstrual bleeding. The Mirena® device releases levonorgestrel (a progestogen).

IUDs have been previously disclosed in a variety of shapes and sizes. See, for example, U.S. Patent and Publications:

US 2005/0045183 A1 to Callister et al. for Methods and Devices issued Mar. 3, 2005;

U.S. Pat. No. 3,407,806 A to Hulka et al for Contraceptive Intra-Uterine Devices issued Oct. 29, 1968;

U.S. Pat. No. 3,902,483 A to Seymour et al. for Intrauterine Device with Locator Means for Indicating Uterine Position of Device issued Sep. 2, 1975;

U.S. Pat. No. 3,937,217 A to Kosenen for Intrauterine Contraceptive Device issued Feb. 10, 1976;

U.S. Pat. No. 4,353,363 A to Quesada for Intrauterine Spermacide issued Oct. 12, 1982;

U.S. Pat. No. 4,359,046 A to Shaw Jr. for IUD Arrangement issued Nov. 16, 1982;

U.S. Pat. No. 4,372,302 A to Akerlund for Instrument for Retrieval of Retracted Threads of Intrauterine Contraceptive Devices issued Feb. 8, 1983;

U.S. Pat. No. 4,381,001 to Shaw Jr. for IUD Arrangement issued Apr. 26, 1983;

U.S. Pat. No. 4,495,934 A to Shaw Jr. for IUD Arrangement issued Jan. 29, 1985;

U.S. Pat. No. 4,830,025 A to Gainutdinova et al. for Intrauterine Contraceptive Device issued May 16, 1989;

U.S. Pat. No. 4,957,119 A to de Nijs for Contraceptive Implant issued Sep. 18, 1990;

U.S. Pat. No. 5,088,505 A to de Nijs for Contraceptive Implant issued Feb. 18, 1992;

U.S. Pat. No. 6,039,968 A to Nabahi for Intravaginal Drug Delivery Device issued Mar. 21, 2000; and

U.S. Pat. No. 7,862,552 B2 to McIntyre et al. for Medical Devices for Treating Urological and Uterine Conditions issued Jan. 4, 2011.

IUDs are typically inserted using an insertion device or instrument. See, for example, U.S. Patents and publications:

U.S. Pat. No. 3,794,025 A to Lerner for Intrauterine Device Saddle Inserter issued Feb. 26, 1974;

U.S. Pat. No. 4,920,727 A to Ristimaki et al. for Cassette System and Apparatus for Manufacturing an Active Agent Liberating Capsule for Subcutnaeous Use issued May 1, 1990;

U.S. Pat. No. 4,949,732 A to Spoon et al. for Apparatus for Insertion and Fixation of an Intra Uterine Contraceptive Device to the Uterine Fundus issued Aug. 21, 1990;

U.S. Pat. No. 5,084,004 A to Ranoux for Process for Intra-Uterine Fertilization in Mammals and Device for Implementation Thereof issued Jan. 28, 1992;

U.S. Pat. No. 5,370,129 A to Diaz et al. for IUD Inserting Apparatus issued Dec. 6, 1994;

U.S. Pat. No. 5,400,804 A to Helle et al. for Method and Equipment for Installing a Medicine Capsule on a Support issued Mar. 28, 1995;

U.S. Pat. No. 5,785,053 A to Macandrew et al. for Inserter for the Positioning of an Intrauterine Device issued Jul. 28, 1998; and

U.S. Pat. No. 3,783,861 A to Abramson for Inserter for Intrauterine Devices issued Jan. 8, 1974.

Other references of interest in the IUS and IUD field include, for example, U.S. Patents and publications:

U.S. Pat. No. 6,056,976 A to Markkula et al. for Elastomer, Its Preparation and Use issued May 2, 2000;

U.S. Pat. No. 6,063,395 A to Markkula et al. for Drug Delivery Device Especially for the Delivery of Progestins and Estrogens issued May 16, 2000;

U.S. Pat. No. 6,103,256 A to Nahabi for Intravaginal Drug Delivery Device issued Aug. 15, 2000;

U.S. Pat. No. 6,117,442 A to Markkula et al. for Drug Delivery Device, Especially for the Delivery of Androgens issued Sep. 12, 2000;

US 2008/0095825 A1 to LaFont for Method for Making a Reservoir Containing an Active Substance Diffused through the Reservoir and Installation Therefor published Apr. 24, 2008; and

US 2013/0014762 A1 to Deckman et al. for Intrauterine Systems, IUD Insertion Devices, and Related Methods and Kits Therefor, published Jan. 17, 2013.

Conventional insertion devices used with IUDs (which includes devices used for IUSs) can cause pain and even loss of consciousness to a patient during the insertion procedure as a result of induction of a vagal reflex response. Conventional insertion devices lack smooth operability and exhibit issues with ease of use. Thus, there exists a need for an insertion device adaptable and configurable for use with IUDs and related methods and kits which reduce patient pain and trauma during the insertion procedure and provides a simple, high-quality, easy-to-use, smoothly operating, economical solution.

### SUMMARY

Disclosed are two-handed IUD insertion devices. The two-handed IUD insertion devices comprise: an elongated sheath having a proximal end and a distal end and a lumen extending between the proximal end and the distal end; a plunger having a proximal end and a distal end with a plurality of notches along its length wherein the plunger is configured to move axially within the lumen of the elongated sheath; a grip secured to the proximal end of the elongated sheath wherein the grip has an interior surface and an exterior surface and a centrally translating member configured to move without rotation towards a central axis to engage one of the plurality of notches on the plunger and an external button that engages the centrally translating member to disengage the centrally translating member away from an engaged notch; a proximally positioned tab handle secured to the proximal end of the plunger. In some configurations, the tab handle has a hinge and/or a textured surface. A flange can be positioned about an exterior of the elongated sheath and visible markings can be provided on the sheath. The two-handed IUD insertion device operates in combination with an intrauterine device (IUD). An elongate member of the IUD comprises a core part containing a polymeric reservoir with an active agent around which a rate limiting polymer membrane has been fitted. The active agent in the reservoir can be a hormone used for the treatment of menopausal troubles or for contraception.

Yet another aspect of the disclosure is directed to two-handed IUD insertion devices comprising: an elongated sheath having a proximal end and a distal end and a lumen extending between the proximal end and the distal end; a plunger having a proximal end and a distal end with a plurality of notches along its length at the proximal end wherein the plunger is configured to move axially within the lumen of the elongated sheath; a grip secured to the proximal end of the elongated sheath; a proximally positioned tab handle having a first member and a second member with a hinge along one edge between the first member and the second member wherein the tab handle is secured to the proximal end of the plunger. In some configurations, the tab handle has a hinge and/or a textured surface. A flange can be positioned about an exterior of the elongated sheath and visible markings can be provided on the sheath. The two-handed IUD insertion device operates in combination with an intrauterine device (IUD). An elongate member of the IUD comprises a core part containing a polymeric reservoir with an active agent around which a polymeric rate controlling membrane has been fitted. The active agent in the reservoir can be a hormone used for the treatment of menopausal troubles or for contraception.

Still another aspect of the disclosure is directed to methods of delivering an IUD comprising: providing a two-handed IUD insertion device having an elongated sheath having a proximal end and a distal end and a lumen extending between the proximal end and the distal end, a plunger having a proximal end and a distal end with a plurality of notches along its length at the proximal end wherein the plunger is configured to move within the lumen of the elongated sheath, a grip secured to the proximal end of the elongated sheath wherein the grip has an interior surface and an exterior surface and a centrally translating member configured to engage one of the plurality of notches on the plunger and an external button that engages the centrally translating member to disengage the centrally translating member away from an engaged notch, and a proximally positioned tab handle secured to the proximal end of the plunger with a t-shaped IUD having two arms positioned within the distal end of the elongated sheath of the two-handed IUD insertion device; advancing the two-handed insertion device into a cervix of a patient until a flange positioned around the elongated sheath is from 1.5 cm to 2.0 cm from an external opening of the cervix; pulling the sheath proximally to release the arms of the IUD; advancing the two-handed insertion device proximally into the cervix until a fundal resistance is detected; and releasing the IUD from the two-handed insertion device. The method can also comprise the step of delivering an active agent used for the treatment of menopausal troubles or for contraception from the IUD.

Yet another aspect of the disclosure is directed to methods of delivering an IUD comprising: providing a two-handed IUD insertion device having an elongated sheath having a proximal end and a distal end and a lumen extending between the proximal end and the distal end; a plunger having a proximal end and a distal end with a plurality of notches along its length wherein the plunger is configured to move axially within the lumen of the elongated sheath; a grip secured to the proximal end of the elongated sheath wherein the grip has an interior surface and an exterior surface and a centrally translating member configured to move without rotation towards a central axis to engage one of the plurality of notches on the plunger and an external button that engages the centrally translating member to disengage the centrally translating member away from an engaged notch; a proximally positioned tab handle secured to the proximal end of the plunger. In some configurations, the tab handle has a hinge and/or a textured surface; advancing the two-handed insertion device into a cervix of a patient until a flange positioned around the elongated sheath is from 1.5 cm to 2.0 cm from an external opening of the cervix; pulling the sheath proximally to release the arms of the IUD; advancing the two-handed insertion device proximally into the cervix until a fundal resistance is detected; and releasing the IUD from the two-handed insertion device. The method can also comprise the step of delivering an active agent used for the treatment of menopausal troubles or for contraception from the IUD.

Another aspect of the disclosure is directed to two-handed IUD insertion devices comprising: an elongated sheath having a proximal end and a distal end and a lumen extending between the proximal end and the distal end; a rotatable plunger having a proximal end and a distal end with a first cross-section at a first orientation, a second cross-section at a second orientation different than the first cross-section orientation and a third cross-section at a third orientation along its length wherein the plunger is configured to move axially within the lumen of the elongated sheath until the cross-section of the plunger changes orientation; a grip secured to the proximal end of the elongated sheath wherein the grip has an aperture shaped to receive the rotatable plunger; a proximally positioned tab handle secured to the proximal end of the plunger. In some configurations, the tab handle has a hinge and/or a textured surface. A flange can be positioned about an exterior of the elongated sheath and visible markings can be provided on the sheath. The two-handed IUD insertion device operates in combination with an intrauterine device (IUD). An elongate member of the IUD comprises a core part containing a polymeric reservoir with an active agent around which a polymeric rate controlling membrane has been fitted. The active agent in the reservoir can be a hormone used for the treatment of menopausal troubles or for contraception.

Yet another aspect of the disclosure is directed to methods of delivering an IUD comprising: providing a two-handed IUD insertion device having an elongated sheath having a proximal end and a distal end and a lumen extending between the proximal end and the distal end; a rotatable plunger having a proximal end and a distal end with a first cross-section at a first orientation, a second cross-section at a second orientation different than the first cross-section orientation and a third cross-section at a third orientation along its length wherein the plunger is configured to move axially within the lumen of the elongated sheath until the cross-section of the plunger changes orientation; a grip secured to the proximal end of the elongated sheath wherein the grip has an aperture shaped to receive the rotatable plunger; a proximally positioned tab handle secured to the proximal end of the plunger; advancing the two-handed insertion device into a cervix of a patient until a flange positioned around the elongated sheath is from 1.5 cm to 2.0 cm from an external opening of the cervix; pulling the sheath proximally to release the arms of the IUD; advancing the two-handed insertion device proximally into the cervix until a fundal resistance is detected; and releasing the IUD from the two-handed insertion device. The method can also comprise the step of delivering an active agent used for the treatment of menopausal troubles or for contraception from the IUD.

Disclosed are two-handed IUD insertion devices. The two-handed IUD insertion devices comprise: an elongated sheath means having a proximal end and a distal end and a lumen extending between the proximal end and the distal end; a plunger means having a proximal end and a distal end with a plurality of notches along its length wherein the plunger means is configured to move axially within the lumen of the elongated sheath means; a grip means secured to the proximal end of the elongated sheath means wherein the grip means has an interior surface and an exterior surface and a centrally translating member configured to move without rotation towards a central axis to engage one of the plurality of notches on the plunger means and an external button means that engages the centrally translating member to disengage the centrally translating member away from an engaged notch; a proximally positioned tab handle means secured to the proximal end of the plunger means. In some configurations, the tab handle means has a hinge and/or a textured surface. A flange means can be positioned about an exterior of the elongated sheath means and visible markings can be provided on the sheath. The two-handed IUD insertion device operates in combination with an intrauterine device (IUD). An elongate member of the IUD comprises a polymeric reservoir with an active agent around which a jacket-like rate controlling polymeric membrane has been fitted. The active agent in the reservoir can be a hormone used for the treatment of menopausal troubles or for contraception.

Yet another aspect of the disclosure is directed to two-handed IUD insertion devices comprising: an elongated sheath means having a proximal end and a distal end and a lumen extending between the proximal end and the distal end; a plunger means having a proximal end and a distal end with a plurality of notches along its length at the proximal end wherein the plunger means is configured to move axially within the lumen of the elongated sheath means; a grip means secured to the proximal end of the elongated sheath means; a proximally positioned tab handle means having a first member and a second member with a hinge along one edge between the first member and the second member wherein the tab handle means is secured to the proximal end of the plunger means. In some configurations, the tab handle means has a hinge and/or a textured surface. A flange means can be positioned about an exterior of the elongated sheath means and visible markings can be provided on the sheath. The two-handed IUD insertion device operates in combination with an intrauterine device (IUD). An elongate member of the IUD comprises a polymeric reservoir with active agent in which a rate controlling membrane has been fitted. The active agent in the reservoir can be a hormone used for the treatment of menopausal troubles or for contraception.

Still another aspect of the disclosure is directed to methods of delivering an IUD comprising: providing a two-handed IUD insertion device having an elongated sheath means having a proximal end and a distal end and a lumen extending between the proximal end and the distal end, a plunger means having a proximal end and a distal end with a plurality of notches along its length at the proximal end wherein the plunger means is configured to move within the lumen of the elongated sheath means, a grip means secured to the proximal end of the elongated sheath means wherein the grip means has an interior surface and an exterior surface and a centrally translating member configured to engage one of the plurality of notches on the plunger means and an external button means that engages the centrally translating member to disengage the centrally translating member away from an engaged notch, and a proximally positioned tab handle means secured to the proximal end of the plunger means with a t-shaped IUD having two arms positioned within the distal end of the elongated sheath means of the two-handed IUD insertion device; advancing the two-handed insertion device into a cervix of a patient until a flange means positioned around the elongated sheath means is from 1.5 cm to 2.0 cm from an external opening of the cervix; pulling the sheath proximally to release the arms of the IUD; advancing the two-handed insertion device proximally into the cervix until a fundal resistance is detected; and releasing the IUD from the two-handed insertion device. The method can also comprise the step of delivering an active agent used for the treatment of menopausal troubles or for contraception from the IUD.

Yet another aspect of the disclosure is directed to methods of delivering an IUD comprising: providing a two-handed IUD insertion device having an elongated sheath means having a proximal end and a distal end and a lumen extending between the proximal end and the distal end; a plunger means having a proximal end and a distal end with a plurality of notches along its length wherein the plunger means is configured to move axially within the lumen of the elongated sheath means; a grip means secured to the proximal end of the elongated sheath means wherein the grip means has an interior surface and an exterior surface and a centrally translating member configured to move without rotation towards a central axis to engage one of the plurality of notches on the plunger means and an external button means that engages the centrally translating member to disengage the centrally translating member away from an engaged notch; a proximally positioned tab handle means secured to the proximal end of the plunger means. In some configurations, the tab handle means has a hinge and/or a textured surface; advancing the two-handed insertion device into a cervix of a patient until a flange means positioned around the elongated sheath means is from 1.5 cm to 2.0 cm from an external opening of the cervix; pulling the sheath proximally to release the arms of the IUD; advancing the two-handed insertion device proximally into the cervix until a fundal resistance is detected; and releasing the IUD from the two-handed insertion device. The method can also comprise the step of delivering an active agent used for the treatment of menopausal troubles or for contraception from the IUD.

Another aspect of the disclosure is directed to two-handed IUD insertion devices comprising: an elongated sheath means having a proximal end and a distal end and a lumen extending between the proximal end and the distal end; a rotatable plunger means having a proximal end and a distal end with a first cross-section at a first orientation, a second cross-section at a second orientation different than the first cross-section orientation and a third cross-section at a third orientation along its length wherein the plunger means is configured to move axially within the lumen of the elongated sheath means until the cross-section of the plunger means changes orientation; a grip means secured to the proximal end of the elongated sheath means wherein the grip means has an aperture shaped to receive the rotatable plunger means; a proximally positioned tab handle means secured to the proximal end of the plunger means. In some configurations, the tab handle means has a hinge and/or a textured surface. A flange means can be positioned about an exterior of the elongated sheath means and visible markings can be provided on the sheath. The two-handed IUD insertion device operates in combination with an intrauterine device (IUD). An elongate member of the IUD comprises a polymeric reservoir containing an active agent around which a rate limiting polymeric membrane has been fitted. The active agent in the reservoir can be a hormone used for the treatment of menopausal troubles or for contraception.

Yet another aspect of the disclosure is directed to methods of delivering an IUD comprising: providing a two-handed IUD insertion device having an elongated sheath means having a proximal end and a distal end and a lumen extending between the proximal end and the distal end; a rotatable plunger means having a proximal end and a distal end with a first cross-section at a first orientation, a second cross-section at a second orientation different than the first cross-section orientation and a third cross-section at a third orientation along its length wherein the plunger means is configured to move axially within the lumen of the elongated sheath means until the cross-section of the plunger means changes orientation; a grip means secured to the proximal end of the elongated sheath means wherein the grip means has an aperture shaped to receive the rotatable plunger means; a proximally positioned tab handle means secured to the proximal end of the plunger means; advancing the two-handed insertion device into a cervix of a patient until a flange means positioned around the elongated sheath means is from 1.5 cm to 2.0 cm from an external opening of the cervix; pulling the sheath proximally to release the arms of the IUD; advancing the two-handed insertion device proximally into the cervix until a fundal resistance is detected; and releasing the IUD from the two-handed insertion device. The method can also comprise the step of delivering an active agent used for the treatment of menopausal troubles or for contraception from the IUD.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1C illustrate a conventional IUD insertion device;

FIG. 2 illustrates a conventional T-shaped IUD;

FIGS. 3A-B illustrates a two handed IUD inserter with a tab; FIG. 3C illustrates a cross-section of the grip;

FIG. 4 illustrates a two-handed IUD inserter with detents along the length to allow threads to be secured;

FIGS. 5A-H illustrate a two-handed IUD inserter with a tab and a retractable grip;

FIGS. 6A-E illustrate details of the tab plunger of the two-handed IUD inserter of FIG. 5;

FIGS. 7A-C illustrate a single-handed IUD inserter;

FIGS. 8A-C illustrate another configuration of a single-handed IUD inserter;

FIGS. 9A-C illustrate another configuration of a single-handed IUD inserter;

FIGS. 10A-B, and 11 illustrate another configuration of a single-handed IUD inserter; and

FIG. 12 illustrates a plurality of mechanical features for the proximal end of an IUD inserter.

### DETAILED DESCRIPTION

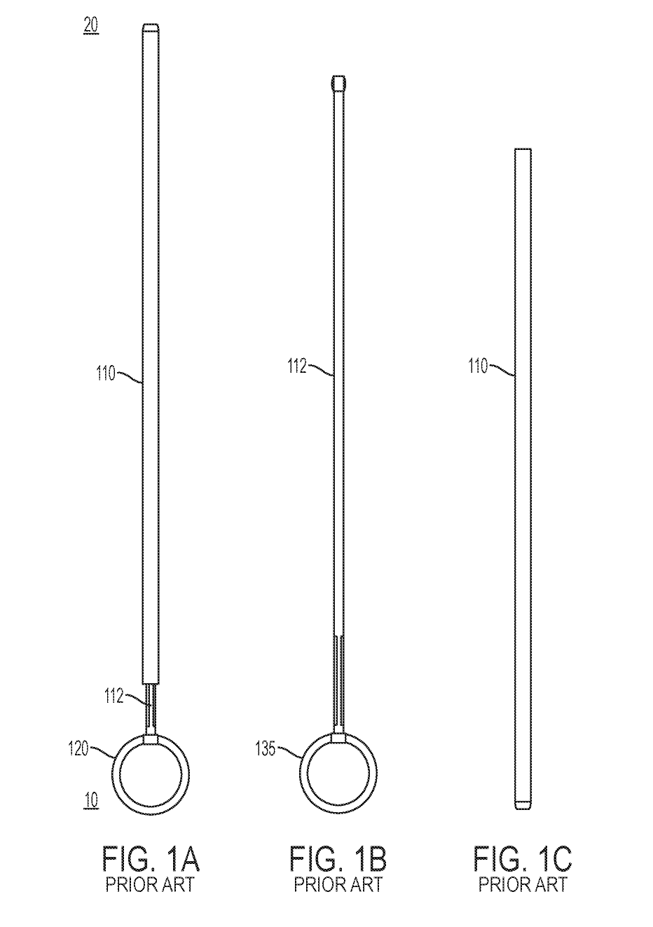

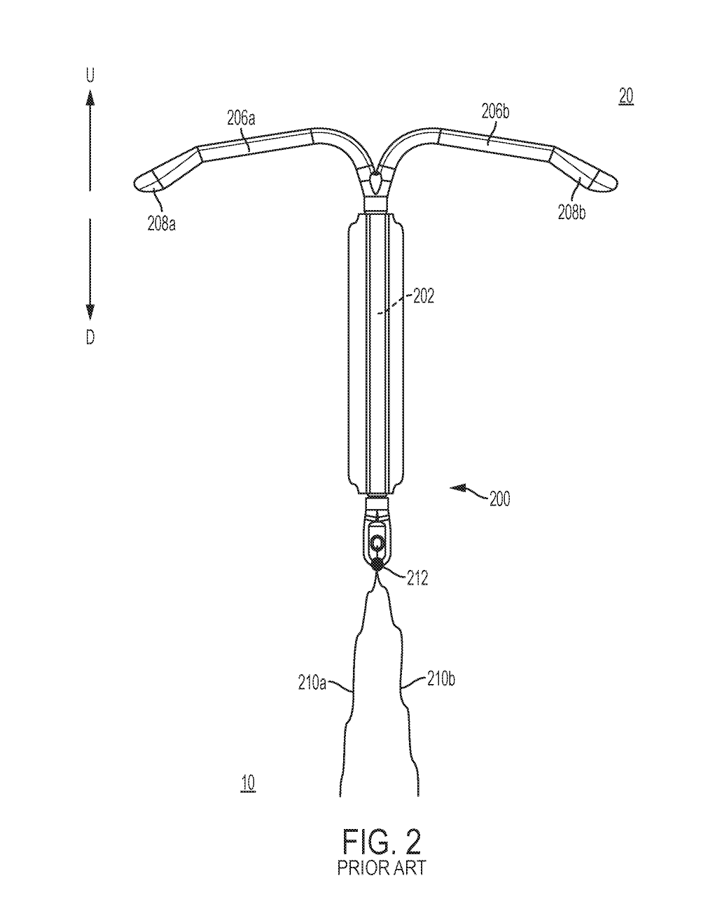

Conventional intrauterine insertion devices include an inserter or insertion device 100 such as the device shown in FIGS. 1A-1C, which includes a sheath 110 or insertion tube having a proximal end 10 and a distal end 20 and a lumen extending between the proximal end and the distal end of the sheath 110 for housing the IUD (shown in FIG. 2), a plunger 112 for pushing the IUD through the sheath 110, and a user interface such as a handle 120 for holding the insertion device. The device shown in FIGS. 1A-1C requires a two-handed operation, whereby the operator or user holds the handle 120 in one hand and the sheath 110 in another hand.

The disclosed insertion devices can, for example, be used with a T-shaped IUD 200, such as the IUD as shown in FIG. 2. IUDs typically have a length of from about 31.90 mm to about 32.22 mm and a width of from about 31.81 mm to about 32.13 mm when the IUD is in the fully deployed position. As will be appreciated by those skilled in the art, the length does not include the knot or strings that may accompany the IUD. The T-shaped IUD 200 comprises an elongated body 202 having a proximal end 10 and a distal end 20. The elongated body 202 can include a coating such as a time-release drug or hormone. The elongated body can be formed from any suitable material, including, but not limited to plastic or copper. At the distal end 20 of the IUD (i.e., the end positioned away from the physician's hand), arms 206a, 206b are attached to or integrally formed with the elongated body 202. The arms 206a, 206b are configurable to fold upward u or downward d to minimize the IUD cross-section such that the IUD can fit into a lumen of the sheath of the insertion device or a tube for insertion through the cervix and into the uterus. Additionally, either or both of the arms 206a, 206b are configurable so that the ends of the tips 208a, 208b are enlarged or bulbous, which can, for example, have a curved, spherical or semi-spherical shape. The tips 208a, 208b of the arms 206a, 206b can be formed such that the arms, when folded upward and pushed together, form a smooth and rounded distal tip. At the proximal end 10 of the t-shaped IUD 200, the t-shaped IUD 200 can further include one or more strings 210a, 210b attached to the IUD. The strings are connectable to the IUD at a connection point 212, e.g., tied in a knot as illustrated.

Although the insertion device is generally described herein with regard to a T-shaped IUD such as the t-shaped IUD 200 shown in FIG. 2, it should be noted that the insertion devices of the present disclosure are adaptable to facilitate insertion of other IUD configurations, as would be appreciated by a person of skill in the art. Moreover, insertion device operation and IUD insertion procedures can include any number of steps corresponding to a desired IUD position. In addition to the features described below, the insertion devices of the present disclosure include IUD position control features which may be advantageous for insertion of IUDs having a variety of configurations. For example, while the IUD insertion procedure described below refers to a three-phase procedure corresponding to three different IUD positions, the insertion device operation procedure can include less than three or more than three steps. Accordingly, the insertion devices can include any number of position control features corresponding to the desired IUD positions. The insertion device of the present disclosure can be used with various conventional IUDs available on the market, including such devices as the T-frame LNg-20 IUD, marketed as Mirena® by Bayer®, as well as the Neo-Safe CuT 380A™ available from Mona-Lisa™.

Insertion device disclosed herein are configurable to operate according to procedural steps which generally mimic commonly known and used procedures for IUD insertion. However, the insertion device of the present disclosure includes improvements in device structure and operation. In another aspect of the disclosed devices, procedural steps for IUD insertion include: (i) pre-insertion insertion device preparation procedures, (ii) a first phase of IUD insertion (also referred to herein as phase 1, position 1, or step 1), (iii) a second phase of IUD insertion (also referred to herein as phase 2, position 2, or step 2), (iv) a third phase of IUD insertion (also referred to herein as phase 3, position 3, or step 3), and (v) post-insertion procedures.

Pre-insertion insertion device preparation procedures can include loading an IUD, such as the IUD illustrated in FIG. 2, into an insertion device, aligning the IUD in-plane with a patient, positioning the IUD in a correct longitudinal position along the length of a sheath of the insertion device, and locking the IUD into a position for insertion. Such pre-insertion insertion device preparation procedures are described in further detail below.

A contraceptive device, which is available on the market and which releases levonorgestrel, consists of a T-shaped IUD 200 having an elongated body 202 fabricated of polyethylene equipped with a reservoir adjusted around it and containing the hormone levonorgestrel. The IUD comprises a polymeric reservoir containing an active agent around which a rate controlling polymeric membrane has been fitted. The active agent includes hormones used for the treatment of menopausal troubles or for contraception. The IUD is sold in sterile packaging together with the inserter with the plunger contained within the protecting tube. The T-shaped IUD 200 is positioned at the forward end of the plunger with the hormone-containing elongate member protected by the tube. The arms 206a, 206b of the transverse member, on the other hand, are expanded in order to prevent fatigue. The strings by which the T-shaped device is retracted towards the outside run between the plunger and the protective tube and end at the end of the handle. The strings 210a, 210b remain attached to the IUD 200 after the IUD has been inserted into the uterus. The strings are trimmed so that the strings extend into the vagina from the opening of the cervix.

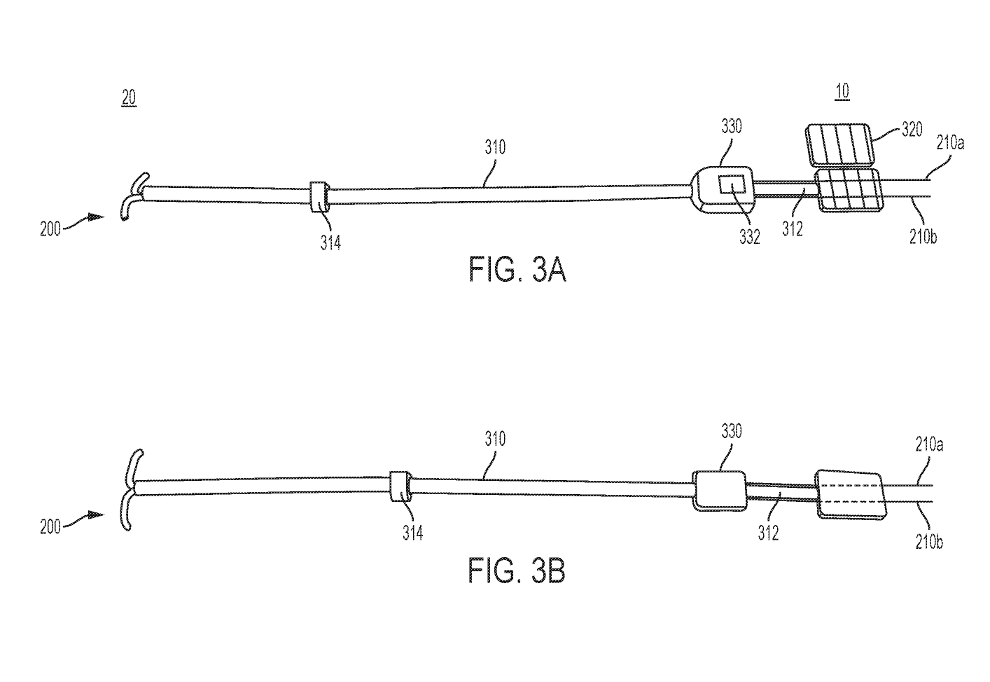

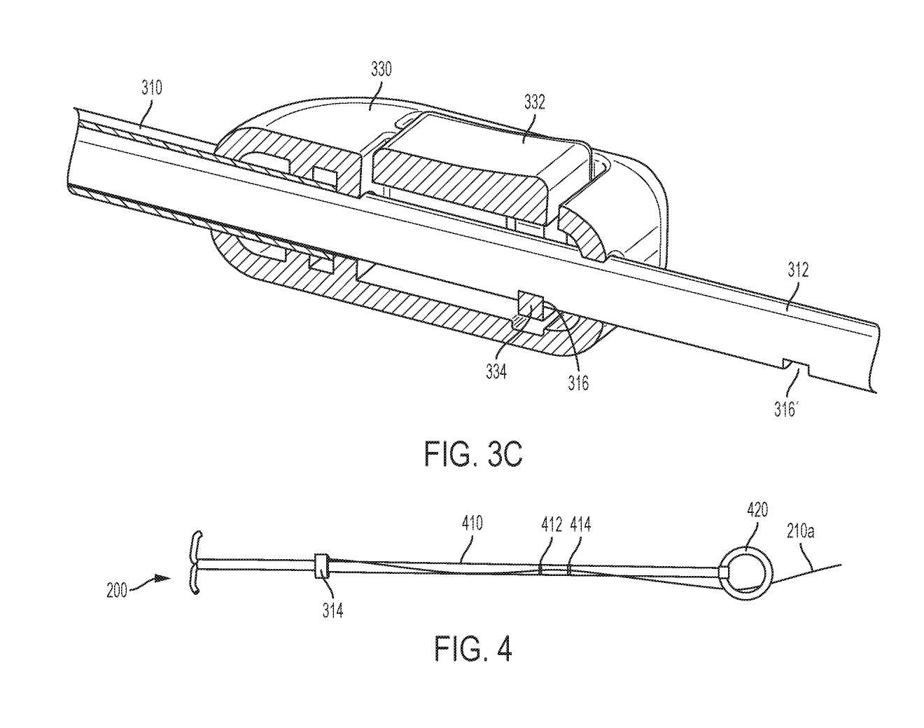

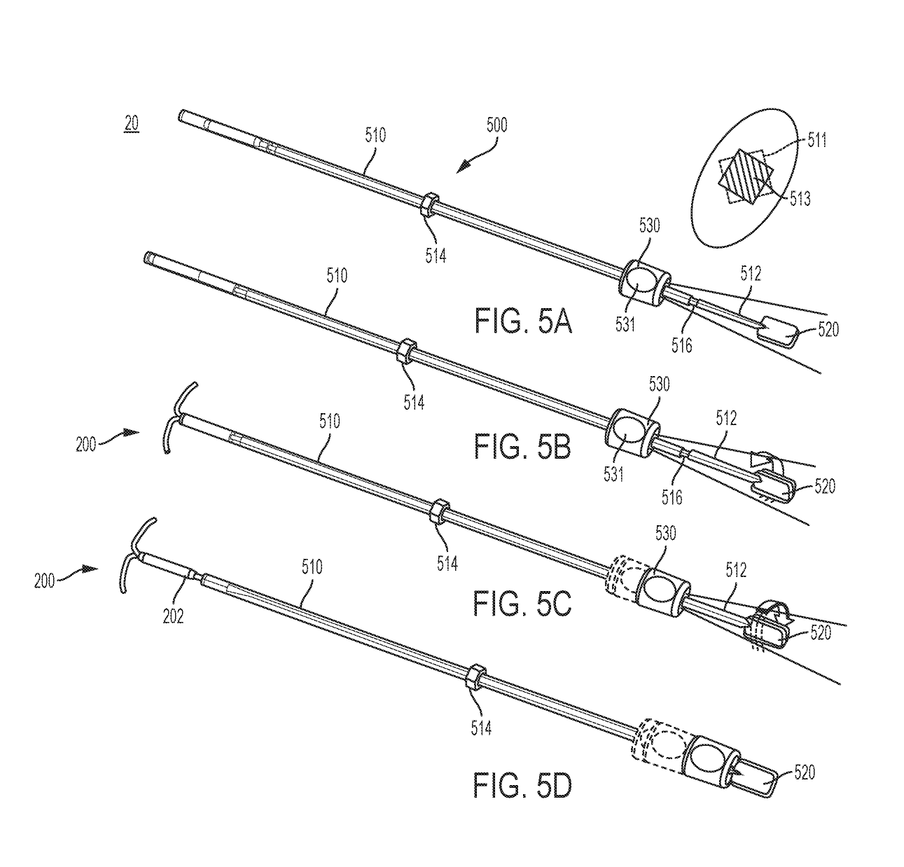

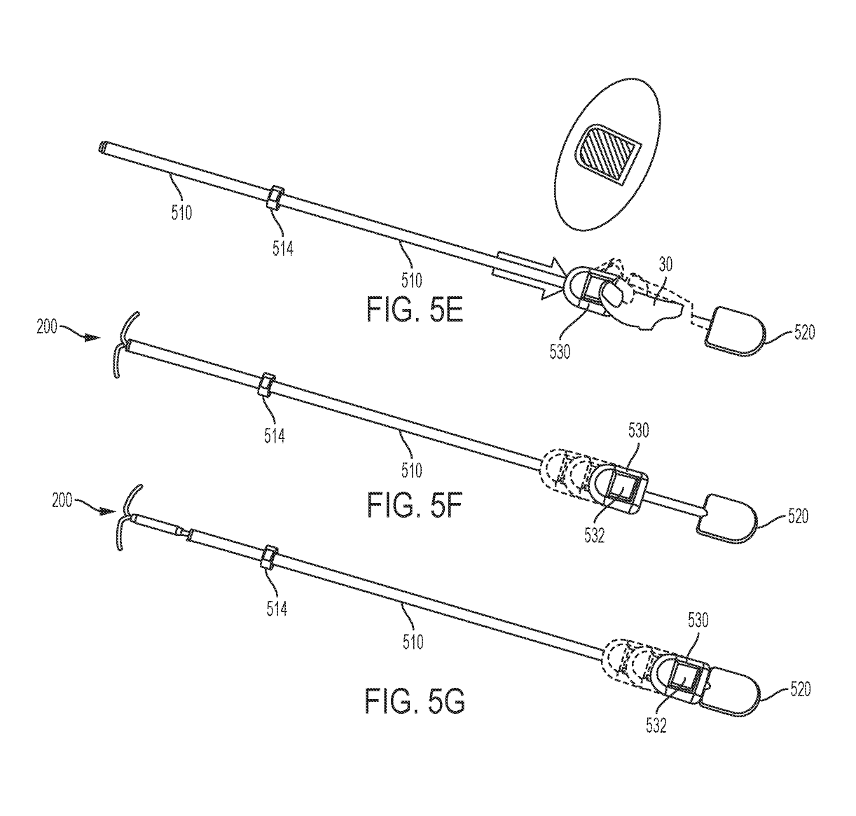

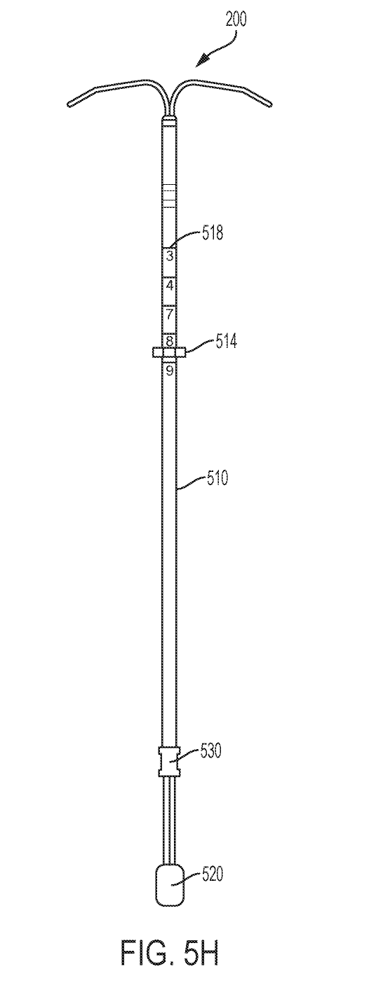

FIGS. 3A-C illustrates a two handed IUD inserter 300 with a tab handle 320 having a proximal end 10 and a distal end 20. The two handed IUD inserter 300 has a sheath 310 which defines a lumen, with a flange 314. The sheath 310 is configured to hold a t-shaped IUD 200 within its lumen at a proximal end 10 prior to insertion and a plunger 312 within the lumen. The plunger 312 acts as a plunger and can be locked in place until the user squeezes a button 332 on a grip 330 that is affixed to a proximal end of the sheath 310 to release the plunger 312. Once the plunger 312 is released the plunger 312 can be pushed into or withdrawn from the lumen of the sheath 310. The tab handle 320 can be hinged 322 to allow the tab handle 320 to fold closed (as shown from FIG. 3A to FIG. 3B). Additionally, texturing 324 can be provided on a surface of the tab handle 320 to engage the IUD threads 210 of the t-shaped IUD 200. In some configurations, the tab handle 320 need not be hinged. Where the tab handle 320 is not hinged, the texturing 324 can be on any or all surfaces of the tab handle 320 Markings can be applied to the plunger 312 or the sheath 310, such as ink markings (shown in FIG. 5H), to provide the user information on depth during the insertion procedure. Flange 314 movement can be achieved using different materials, geometry or processing.

Turning to FIG. 3C, a cut-away of the grip 330 engaging the sheath 310 and plunger 312 is shown. The plunger 312 has two or more notches 316, 316′ along its length which are configured to engage a lever 334 within the body of the grip 330. When the lever 334 engages one of the two or more notches 316 as the plunger 312 is moved (proximally or distally) through the sheath 310, the lever 334 translates up and into one of the two or more notches 316 which results restricting the movement of the plunger 312. To release the plunger 312, the user presses the button 332 on a surface of the grip 330 which then translates the lever 334 downward and away from one of the two or more notches 316, thereby allowing the plunger 312 to move within the sheath 310. While pressure is applied to the button 332, the sheath can freely move proximally or distally. Once pressure is released from the button 332, the sheath can freely move until the lever 334 engages another notch of the two or more notches 316 on the plunger 312.

FIG. 4 illustrates a two-handed IUD inserter 400 with one or more indentations 412, 414 along the length of the sheath 410. The indentations 412, 414 allow IUD threads 210 (from FIG. 2) to be secured while the IUD is being deployed.

FIGS. 5A-H illustrate configurations for two-handed IUD inserter 500 with a tab handle 520 at the proximal end of the plunger 512, a grip 530 and a flange 514 positioned around a sheath 510.

FIGS. 5A-D show the two-handed IUD inserter 500 at various stages of inserting the t-shaped IUD 200 with the tab handle 520 rotating about an axis a corresponding to a length of the two-handed IUD inserter 500. The tab handle 520 rotates (FIG. 5B) to align a cross-section 511 of the sheath 510 with the cross-section 513 of the plunger 512. Once the shapes are aligned or keyed, the plunger 512 can be advanced distally into the lumen of the sheath 510. As the plunger 512 is advanced into the lumen of the sheath 510, the arms of the t-shaped IUD 200 is released from the distal end 20 of the two-handed IUD inserter 500 as shown in FIG. 5C, and then the t-shaped IUD 200 is released entirely from the distal end 20 of the two-handed IUD inserter 500. Twisting the tab handle 520 clockwise or counter-clockwise allows the cross-section 511 of the sheath 510 to line up with the cross-section 513 of the plunger 512 to allow the plunger 512 to be advanced into the sheath 510. As will be appreciated by those skilled in the art, the cross-sectional shape of the plunger 512 can vary along its length to provide a stopping mechanism for advancement of the plunger 512 through the sheath 510 at discrete times during the insertion process. A depression 531 can be provided on the grip 530 to improve the user's engagement with the device during use.

In use, the two-handed IUD inserter 500 of FIGS. 5A-D with a tab handle 520 is advanced into the cervix with the IUD positioned with the arms positioned within the distal end of the sheath. The two-handed IUD inserter 500 is inserted into the cervix until the flange 514 is 1.5-2.0 cm from the cervix. At this point the cross-section of the plunger 512 changes to cause distal motion of the plunger to stop. The tab handle 520 is then rotated to align the cross-section of the plunger 512 with the aperture of the grip 530 and the plunger 512 is advanced to release the arms of the IUD. At this point the cross-section of the plunger 512 changes to cause continued distal motion of the plunger to stop. The tab handle 520 is then rotated again to align the cross-section of the plunger 512 with the aperture of the grip 530 and the plunger 512 is advanced until the flange meets the cervix and the user feels fundal resistance. At this point, the IUD is released and the inserted is withdrawn from the cervix. As shown in the call-out, the cross-section of the plunger is square along its length, but the square cross-section changes relative to a square cross-section at another location along the length of the plunger.

FIGS. 5E-G show additional details of the interaction between the plunger 512, the grip 530, the flange 514, and the tab handle 520, with the finger 30 of a user on the grip 530 of the cross-section shown in FIGS. 3A-C and discussed above. Instead of the oval shaped depression shown in FIGS. 5A-D, a button 532 is provided. Depressing the button 532 allow the plunger 512 to be advanced into the sheath 510. As with the design shown in FIGS. 5A-D, the tab handle is rigidly affixed to the plunger 512 and the grip 530 is rigidly affixed to the sheath 510. The cross-section of the plunger and grip is rounded at one end and flat at a second end.

In use, the two-handed IUD inserter 500 of FIGS. 5E-G with a tab handle 520 is advanced into the cervix with the IUD positioned with the arms positioned within the distal end of the sheath. The two-handed IUD inserter 500 is inserted into the cervix until the flange 514 is 1.5-2.0 cm from the cervix. At this point distal motion of the plunger stops. The button 532 on the tab handle 520 is then depressed which allows the plunger 512 to be advanced distally until the arms of the IUD 200 are released. Continued distal motion of the plunger then stops. The button 532 on the tab handle 520 is then depressed again and the plunger 512 is advanced until the flange meets the cervix and the user feels fundal resistance. At this point, the IUD is released and the inserter is withdrawn from the cervix.

Turning to FIG. 511, additional features of the sheath 510 can include markings 516 along its length to indicate distance (e.g., 1 cm, 2 cm, 3 cm . . . 10 cm).

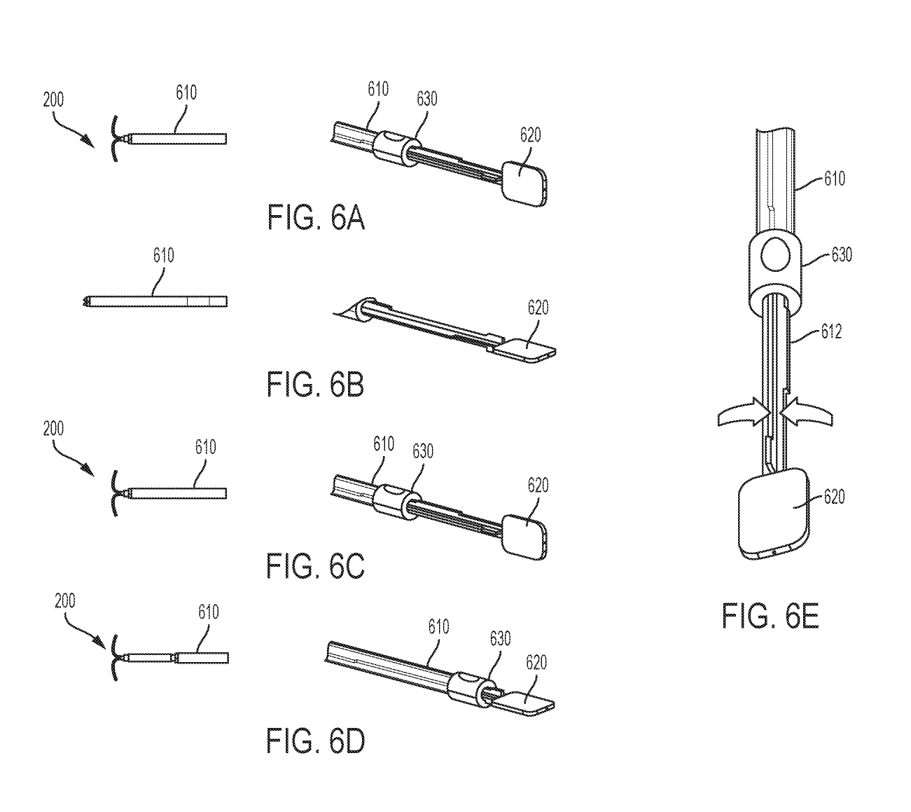

FIGS. 6A-E illustrate additional configuration options for the plunger 512 of the two-handed IUD inserter of FIGS. 5A-D. The grip 630 is connected to the sheath 610 and is configured with keyhole indentions which are shaped to receive a corresponding detents on the plunger 612 and allow for axial movement of the plunger 612 when the orientation of the detents on the plunger and the keyhole of the aperture in the grip 630 are aligned.

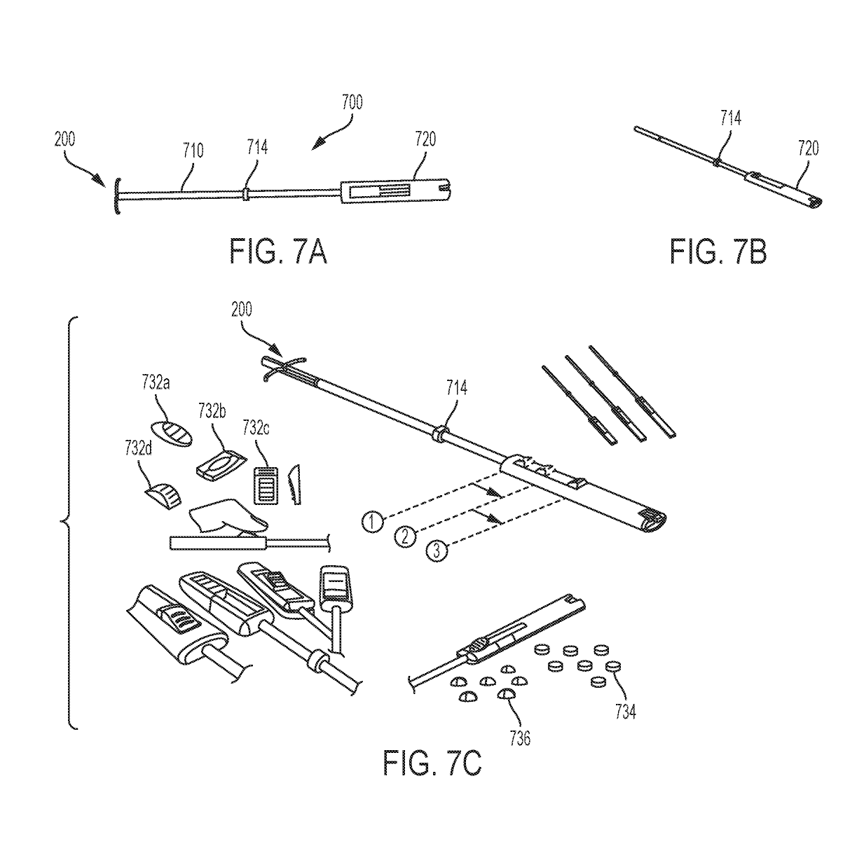

FIGS. 7A-C illustrate a single-handed IUD inserter 700. The single-handed IUD inserter 700 has a sheath 710, a flange 714, a handle 720, and a button 732 that moves within a recess formed in the handle to control release of the t-shaped IUD 200. The button moves, for example, from position 1, to position 2, to position 3. The button 732 can have a variety of shapes 732a, 732b, 732c, 732d and can include various styles of surface texturing 734, 736 to facilitate engaging a user's finger during operation. The single-handed IUD inserter 700 can come in different sizes 700a, 700b, 700c.

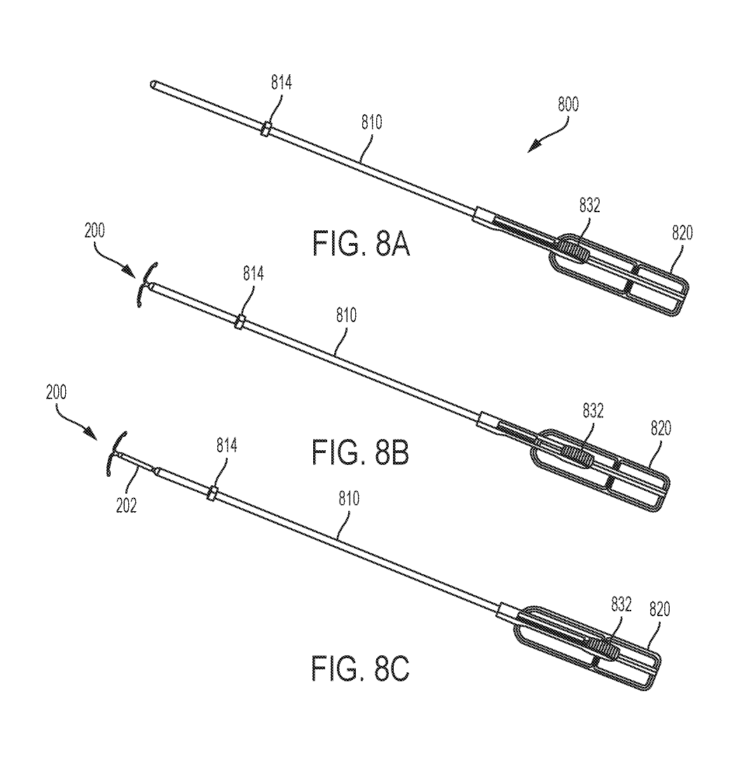

FIGS. 8A-C illustrate another configuration of a single-handed IUD inserter 800 for use with an IUD 200. The single-handed inserter 800 has a sheath 810, a flange 814, and a handle 820. The handle 820 has an elongated frame 822 which defines an open space 824, and a first cross-member 826 which is positioned midway along the length of a proximal end and a distal end and an elongated central cross-member which is positioned in alignment with the axis formed by the sheath 810. The handle 810 is affixed to the plunger and the button 832 engages the sheath 810 so that the handle can move towards and away from the button in operation.

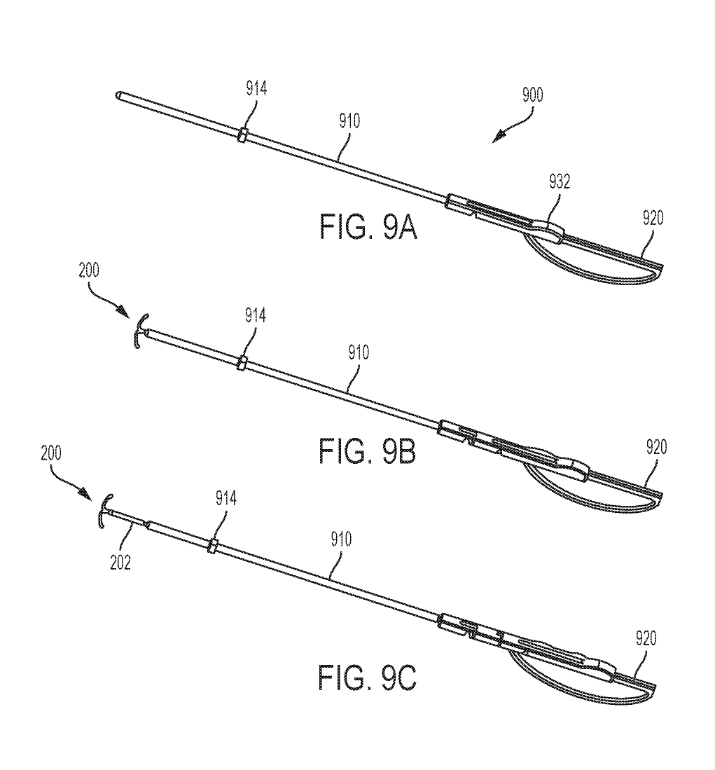

FIGS. 9A-C illustrate another configuration of a single-handed IUD inserter 900 for use with an IUD 200. The single-handed inserter 900 has a sheath 910, a flange 914, and a handle 920. The handle 920 has an elongated flat upper end which defines an open space between the elongated flat upper end and the curved lower member. The handle 910 is affixed to the plunger and the button 932 engages the sheath 910 so that the handle can move towards and away from the button in operation.

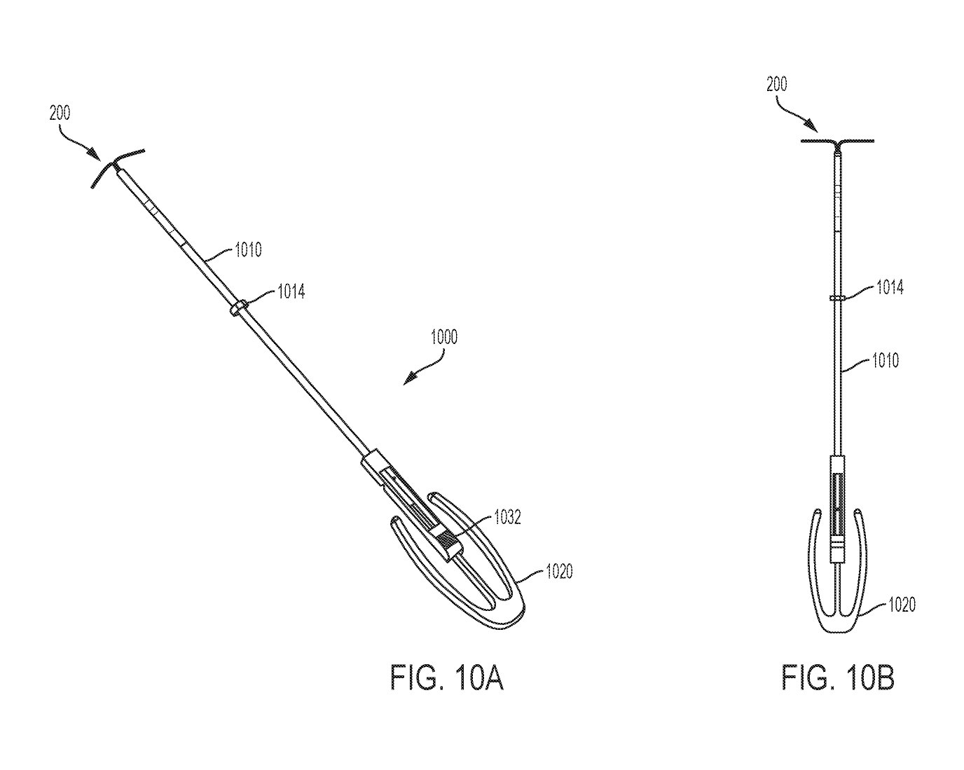

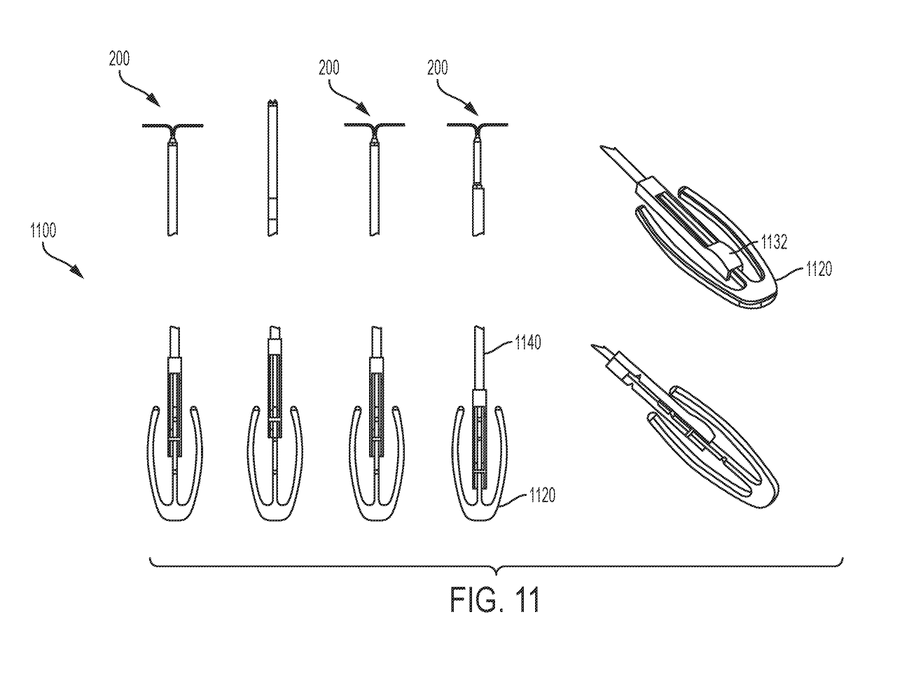

FIGS. 10A-B, and 11 illustrate another configuration of a single-handed IUD inserter 1000, 1100 for use with an IUD 200. The single-handed inserters 1000, 1100 have a sheath 1010, 1110, a flange 1014, 1114, and a handle 1020, 1120. The handle 1020 in FIG. 10 has an elongated central member with two curved members extending away from the proximal end of the central member and then passing adjacent the elongated central member such that the handle 1020 has an M shape in one plane. A corresponding open space exists between the central member and the two curved member. Additionally, the two curved members can curve towards a central axis formed by the central member. The handle 1010 is affixed to the plunger and the button 1032 engages the sheath 1010 so that the handle can move towards and away from the button in operation.

The handle 1120 in FIG. 11 has an elongated central member with two curved members extending away from the proximal end of the central member and then passing adjacent the elongated central member such that the handle 1120 has an M shape in one plane. A corresponding open space exists between the central member and the two curved member. Additionally, the two curved members can curve towards a central axis formed by the central member. The handle 1110 is affixed to the plunger and the button 1132 engages the sheath 1110 so that the handle can move towards and away from the button in operation. A series of notches can be provided along the length of the central member to control the movement of the handle relative to the sheath during operation.

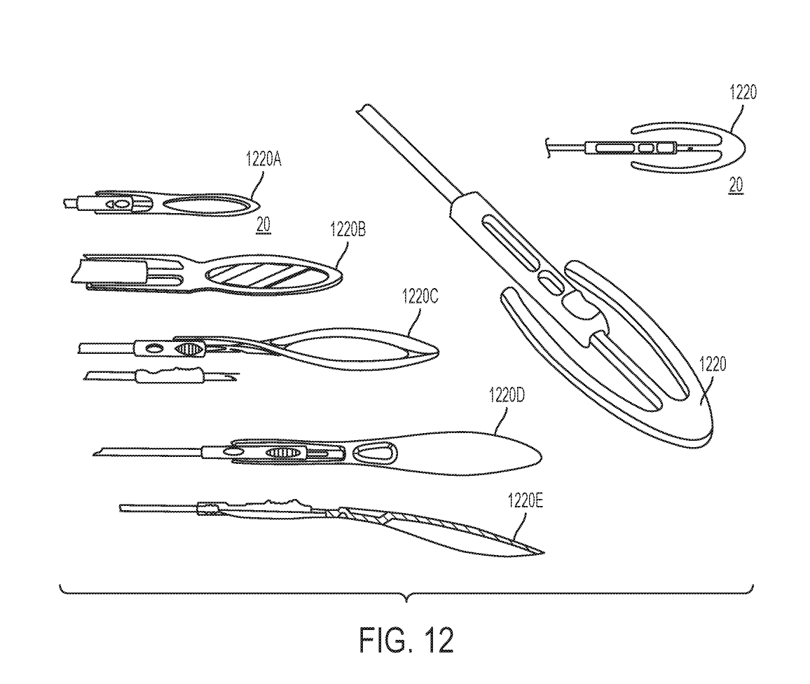

FIG. 12 illustrates a plurality of mechanical features for the proximal end of an IUD inserter 1200. A variety of shapes of the proximal most portion of the handle can be provided. In a first handle 1220a the handle is elongated with two ides and an aperture formed along a portion of its length. The second handle 1220b can be configured similar to the first handle 1220a with the addition of cross members in the aperture. The cross-members can be perpendicular or positioned at an angle. The third handle 1220c has a similar configuration to the first handle 1220a but is twisted about an axis corresponding to the sheath. The fourth handle 1220d has a solid bulbous end with a small aperture positioned proximally. The fourth handle 1220d can further be bent away from an axis corresponding to the sheath as shown in the fifth handle 1220e.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A two-handed IUD insertion device comprising:
an elongated sheath having a proximal end and a distal end and a lumen extending between the proximal end and the distal end;
a plunger having a proximal end and a distal end with a plurality of notches along its length wherein the plunger is configured to move axially within the lumen of the elongated sheath;
a grip secured to the proximal end of the elongated sheath wherein the grip has an interior surface and an exterior surface and a centrally translating member configured to move without rotation towards a central axis to engage one of the plurality of notches on the plunger and an external button that engages the centrally translating member to disengage the centrally translating member away from an engaged notch;
a proximally positioned tab handle secured to the proximal end of the plunger.

2. The two-handed IUD insertion device of claim 1 wherein the tab handle has a hinge.

3. The two-handed IUD insertion device of claim 1 wherein the tab handle has a textured surface.

4. The two-handed IUD insertion device of claim 1 further comprising a flange positioned about an exterior of the elongated sheath.

5. The two-handed IUD insertion device of claim 1 further comprising visible markings on the sheath.

6. The two-handed IUD insertion device according to claim 1, in combination with an intrauterine device (IUD).

7. The two-handed IUD insertion device according to claim 6, wherein an elongate member of the IUD comprises a polymeric reservoir containing an active agent around which a rate controlling polymeric membrane has been fitted.

8. The combination according to claim 7, wherein the active agent is a hormone used for the treatment of menopausal troubles or for contraception.

9. A two-handed IUD insertion device comprising:
an elongated sheath having a proximal end and a distal end and a lumen extending between the proximal end and the distal end;
a plunger having a proximal end and a distal end with a plurality of notches along its length at the proximal end wherein the plunger is configured to move axially within the lumen of the elongated sheath;
a grip secured to the proximal end of the elongated sheath;
a proximally positioned tab handle having a first member and a second member with a hinge along one edge between the first member and the second member wherein the tab handle is secured to the proximal end of the plunger.

10. The two-handed IUD insertion device of claim 9 wherein the tab handle has a hinge.

11. The two-handed IUD insertion device of claim 9 wherein the tab handle has a textured surface.

12. The two-handed IUD insertion device of claim 9 further comprising a flange positioned about an exterior of the elongated sheath.

13. The two-handed IUD insertion device of claim 9 further comprising visible markings on the sheath.

14. The two-handed IUD insertion device according to claim 9, in combination with an intrauterine device (IUD).

15. The two-handed IUD insertion device according to claim 14, wherein an elongate member of the IUD comprises a core part around which a jacket-like polymeric reservoir containing an active agent has been fitted.

16. The combination according to claim 14, wherein the active agent is a hormone used for the treatment of menopausal troubles or for contraception.

17. A method of delivering an IUD comprising:
providing a two-handed IUD insertion device having an elongated sheath having a proximal end and a distal end and a lumen extending between the proximal end and the distal end, a plunger having a proximal end and a distal end with a plurality of notches along its length at the proximal end wherein the plunger is configured to move within the lumen of the elongated sheath, a grip secured to the proximal end of the elongated sheath wherein the grip has an interior surface and an exterior surface and a centrally translating member configured to engage one of the plurality of notches on the plunger and an external button that engages the centrally translating member to disengage the centrally translating member away from an engaged notch, and a proximally positioned tab handle secured to the proximal end of the plunger with a t-shaped IUD having two arms positioned within the distal end of the elongated sheath of the two-handed IUD insertion device;
advancing the two-handed insertion device into a cervix of a patient until a flange positioned around the elongated sheath is from 1.5 cm to 2.0 cm from an external opening of the cervix;
pulling the sheath proximally to release the arms of the IUD;
advancing the two-handed insertion device proximally into the cervix until a fundal resistance is detected; and
releasing the IUD from the two-handed insertion device.

18. The method of claim 17 further comprising the step of delivering an active agent used for the treatment of menopausal troubles or for contraception from the IUD.

19. A two-handed IUD insertion device comprising:
an elongated sheath having a proximal end and a distal end and a lumen extending between the proximal end and the distal end;
a rotatable plunger having a proximal end and a distal end with a first cross-section at a first orientation, a second cross-section at a second orientation different than the first cross-section orientation and a third cross-section at a third orientation along its length wherein the plunger is configured to move axially within the lumen of the elongated sheath until the cross-section of the plunger changes orientation;
a grip secured to the proximal end of the elongated sheath wherein the grip has an aperture shaped to receive the rotatable plunger;
a proximally positioned tab handle secured to the proximal end of the plunger.

20. The two-handed IUD insertion device of claim 19 wherein the tab handle has a hinge.

21. The two-handed IUD insertion device of claim 19 wherein the tab handle has a textured surface.

22. The two-handed IUD insertion device of claim 19 further comprising a flange positioned about an exterior of the elongated sheath.

23. The two-handed IUD insertion device of claim 19 further comprising visible markings on the sheath.

24. The two-handed IUD insertion device according to claim 19, in combination with an intrauterine device (IUD).

25. The two-handed IUD insertion device according to claim 24, wherein an elongate member of the IUD comprises a core part around which a jacket-like polymeric reservoir containing an active agent has been fitted.

26. The combination according to claim 25, wherein the active agent is a hormone used for the treatment of menopausal troubles or for contraception.
